(19) United States
(12) Patent Application Publication
(10) Pub. No.: US 2026/0348931 A1
(43) Pub. Date: Nov. 5, 2026

(54) Concentration Sensors and Methods

(71) Applicant: Schaeffler Technologies AG & Co. KG, Herzogenaurach (DE)

(72) Inventors: Stephan Schürer, Regensburg (DE); Andreas Meyer, Zell (DE); Wolfgang Lauerer, Laaber (DE); Thorsten Knittel, Pentling (DE)

(73) Assignee: Schaeffler Technologies AG & Co. KG, Herzogenaurach (DE)

(21) Appl. No.: 19/140,081

(22) PCT Filed: Dec. 18, 2023
(86) PCT No.: PCT/EP2023/086262

§ 371 Date: Jun. 17, 2025

(30) Foreign Application Priority Data
Dec. 21, 2022 (DE) 10 2022 214 190.3

(51) Int. Cl. G01N 33/00

(52) U.S. Cl. CPC G01N 33/0006

(57) ABSTRACT

Various embodiments of the teachings herein include a method for diagnosing y a gas concentration sensor configured to determine a concentration of a gas using a thermal conductivity and/or thermal capacity of the gas, wherein the gas concentration sensor comprises a heater and a temperature sensor field with a plurality of regions and at least one temperature sensor per region. An example includes: generating a heat flow with the heater; determining a temperature distribution on the temperature sensor field using the temperature sensors of the respective regions; and diagnosing the gas concentration sensor using the determined temperature distribution.

### CROSS-REFERENCE TO RELATED APPLICATIONS

This application a U.S. National Stage Application of International Application No. PCT/EP2023/086262 filed Dec. 18, 2023, which designates the United States of America, and claims priority to DE Application No. 10 2022 214 190.3 filed Dec. 21, 2022, the contents of which are hereby incorporated by reference in their entirety.

### TECHNICAL FIELD

The present disclosure relates to gas concentration sensors. Various embodiments of the teachings herein include methods and/or systems for sensing a gas concentration.

### BACKGROUND

Documents U.S. Pat. No. 10,112,486 B2, DE 10 2019 204 284 A1, JP 2019 128 182 A, U.S. Pat. No. 9,027,386 B2 and US 2009 00 61 261 A1 disclose gas sensors. One disadvantage of the devices known from the prior art, in particular, is that diagnosing such gas sensors is only possible with correspondingly high additional technical effort for diagnostic means.

### SUMMARY

Teachings of the present disclosure include systems and/or methods characterized in particular by simple and/or cost-effective implementation. For example, some embodiments include a method for diagnosing a gas concentration sensor, wherein the gas concentration sensor is designed to determine the gas concentration of a gas on the basis of the thermal conductivity and/or the thermal capacity of the gas, wherein the gas concentration sensor Comprises a heater and a temperature sensor field, wherein the temperature sensor field has a plurality of regions with at least one temperature sensor per region, comprising: generating a heat flow by means of the heater, determining a temperature distribution on the temperature sensor field by means of the temperature sensors of the respective regions, and diagnosing the gas concentration sensor on the basis of the determined temperature distribution.

In some embodiments, the method comprises the following: determining a first temperature measurement value by means of a temperature sensor of a first region, determining a second temperature measurement value by means of a temperature sensor of a second region, determining a temperature measurement value difference from the determined temperature measurement values, and determining the temperature distribution on the basis of the temperature measurement value difference.

In some embodiments, the method comprises detecting a malfunction or an interference source of the gas concentration sensor if the determined temperature measurement value difference exceeds a predefined threshold value.

In some embodiments, the threshold value depends on the position of the temperature sensors relative to each other and/or the position of the temperature sensors relative to the heater and/or the distance of the temperature sensors relative to the heater and/or the distance of the temperature sensors relative to each other.

In some embodiments, a diagnosis signal is generated on the basis of the determined temperature distribution.

In some embodiments, a cleaning method for cleaning the gas concentration sensor is initialized the determined temperature measurement value difference exceeds a, the or a further predefined threshold value.

In some embodiments, the temperature sensors are designed as thermoelectric temperature sensors.

In some embodiments, the temperature sensor field is arranged in a temperature sensor field plane, wherein the heater is intersected by a subdivision plane running perpendicular to the temperature sensor field plane, wherein the regions of the temperature sensor field plane are arranged on one side and on the other side of the subdivision plane.

In some embodiments, the heater is intersected by a further subdivision plane running perpendicular to the temperature sensor field plane and perpendicular to the first subdivision plane, wherein the regions of the temperature sensor field plane are arranged on one side and on the other side of the further subdivision plane.

In some embodiments, the gas flows in the extension direction of the subdivision plane or in the extension direction of the first subdivision plane along the heater and/or the temperature sensor field.

In some embodiments, the first region is arranged on one side of the or the first subdivision plane, wherein the second region is arranged on the other side of the or the first subdivision plane.

In some embodiments, the first and second regions are arranged on the same side of the second subdivision plane.

In some embodiments, the first and second regions are two regions separated from each other by at least one subdivision plane.

As another example, some embodiments include a gas concentration sensor having a heater, a temperature sensor field which has a plurality of regions, wherein each region comprises at least one temperature sensor, and means adapted to carry out one or more of the methods described herein.

As another example, some embodiments include a computer program comprising commands that cause a device to carry out one or more of the methods described herein.

As another example, some embodiments include a computer-readable medium storing a computer program as described herein.

### BRIEF DESCRIPTION OF THE DRAWINGS

Developments of the present teachings are described in the dependent claims and in the description of the figures that follows. The teachings are explained in detail below with reference to exemplary embodiments and the drawings, in which:

FIG. 1 shows a motor vehicle incorporating teachings of the present disclosure;

FIG. 2 shows an example method incorporating teachings of the present disclosure;

FIG. 3 shows an illustration of three planes; and

FIG. 4 shows a gas concentration sensor incorporating teachings of the present disclosure.

### DETAILED DESCRIPTION

Some embodiments of the teachings herein include a method for diagnosing a gas concentration sensor. The gas concentration sensor is designed to determine the gas concentration of a gas on the basis of the thermal conductivity and/or the thermal capacity of the gas, wherein the gas concentration sensor comprises a heater and a temperature sensor field, wherein the temperature sensor field has a plurality of regions with at least one temperature sensor per region. The method comprises the steps of generating a heat flow by means of the heater, determining a temperature distribution on the temperature sensor field by means of the temperature sensors of the respective regions, and diagnosing the gas concentration sensor on the basis of the determined temperature distribution. For this purpose, it is preferred if the heater influences or can influence the temperature or temperature distribution by means of the heat flow it generates.

This makes it possible to perform a cost-effective and simple diagnosis of the gas concentration sensor. In other words, a homogeneous temperature distribution means that a properly functioning gas concentration sensor can be assumed, while an inhomogeneous temperature distribution means a defective gas concentration sensor. Instead of assuming a defective gas concentration sensor, it is also conceivable to assume an interference source. Such an interference source can be formed, for example, by a water droplet or a dirt particle on the temperature sensor field or in the immediate vicinity of one of the temperature sensors. A water droplet can form, for example, by condensation.

Such gas concentration sensors are based in particular on the fact that they make use of the thermal conductivity and/or the thermal capacity of the gas or gas mixture in order to determine the concentration of, for example, hydrogen or nitrogen within the gas or gas mixture. For this purpose, for example, the heater is activated and its surroundings are heated up. The time that elapses before a certain temperature can be determined by means of the temperature sensors is a characteristic value for the gas concentration of, for example, hydrogen or nitrogen within the gas or the gas mixture to be measured. Alternatively, it is conceivable to use the temperature determined by means of the temperature sensors as a characteristic value for the gas concentration. Both of these characteristic values depend on the thermal conductivity and/or the thermal capacity of the gas or gas mixture. The thermal conductivity and/or the thermal capacity of the gas or gas mixture in turn depends on the gas concentration, for example the gas concentration of hydrogen or nitrogen within the gas or gas mixture. In other words, the gas concentration can be determined in this way.

In some embodiments, the gas concentration sensor is designed to determine the gas concentration of hydrogen and/or nitrogen of a gas or gas mixture or a corresponding gas flow or gas mixture flow.

In some embodiments, the gas is a gas mixture or a corresponding flow, which in particular comprises hydrogen and/or nitrogen.

In some embodiments, the determination is a measurement carried out by means of the gas concentration sensor.

In some embodiments, a plurality of temperature sensors or a large number of temperature sensors are provided per region. This allows a higher resolution of the temperature distribution to be determined by means of the temperature sensors. In some embodiments, the same number of temperature sensors are provided for each region. This allows the temperature distribution on the temperature sensor field to be determined in a uniform manner.

In some embodiments, the heater is an electric heater or an electric heating element. n some embodiments, it is a resistance heating element or a PTC heating element. In some embodiments, the temperature measurement values are available as electronic signals, for example as analog or digital signals. Such a signal may correspond to a temperature value in Kelvin or degrees Celsius.

In some embodiments, the method comprises determining a first temperature measurement value by means of a temperature sensor of a first region, determining a second temperature measurement value by means of a temperature sensor of a second region, determining a temperature measurement value difference from the determined temperature measurement values, and determining the temperature distribution on the basis of the temperature measurement value difference. In other words, the basic idea behind the method is to compare the prevailing temperature distribution with the determined temperature distribution.

In some embodiments, the temperature measurement values are available as absolute values or differential values. A differential value is understood in particular to mean the difference relative to a reference value. The reference value is preferably to be understood as the temperature of the heater or the ambient temperature of the gas concentration sensor.

In some embodiments, the difference in temperature measurement values is the difference between the determined temperature measurement values.

In some embodiments, the method comprises detecting a malfunction or an interference source of the gas concentration sensor if the determined temperature measurement value difference exceeds a predefined threshold value. In some embodiments, falling below the temperature measurement value difference or a further temperature measurement value difference can lead to the detection of a malfunction. In some embodiments, the temperature measurement value difference is an absolute value.

In some embodiments, a correct operation of the gas concentration sensor is detected if the measured temperature difference corresponds to the threshold value or is below the threshold value. In some embodiments, in such a case it can be detected that no interference sources are present.

In some embodiments, the threshold value for the temperature measurement value difference is a maximum of 15%, 10%, 5%, 3%, 1%, 0.05% or 0.01% of the lowest or highest temperature measurement value in the temperature unit Kelvin, which is used to determine the temperature measurement value difference.

In some embodiments, the threshold value depends on the position of the temperature sensors relative to each other and/or the position of the temperature sensors relative to the heater and/or the distance of the temperature sensors relative to the heater and/or the distance of the temperature sensors relative to each other. Such a dependency is necessary in particular if the temperature sensors, by means of which the temperature measurement values and the temperature measurement value difference are determined, are unevenly positioned or spaced apart relative to the heater.

In some embodiments, the regions and/or the temperature sensors of the regions are arranged such that in the case of a uniform temperature distribution they determine the same temperature measurement values and thus a uniform temperature distribution.

In some embodiments, a diagnosis signal is generated on the basis of the determined temperature distribution. Such a diagnosis signal may be provided as an electronic signal, for example as an analog or digital signal. The diagnosis signal may be forwarded via a signal line to a control unit or an evaluation unit, which can be part of a control unit. Such a control unit or such an evaluation unit is able to generate an error message by means of the received diagnosis signal. Such an error message can, for example, be stored on a or the control unit and read out at a later time, for example during an inspection or as part of a repair measure.

In some embodiments, the diagnosis signal comprises information about the malfunction of the gas concentration sensor.

In some embodiments, the diagnosis signal is only generated if the determined temperature measurement value difference exceeds the predefined threshold value. In other words, in such a case, a diagnosis signal is only generated if a malfunction or an interference source of the gas concentration sensor is detected. This is especially useful because it is usually assumed that the gas concentration sensor is functioning in an error-free manner.

In some embodiments, a cleaning method for cleaning the gas concentration sensor is initialized if the determined temperature measurement value difference exceeds a, the or a further predefined threshold value. In other words, a cleaning method is started in order to clean the gas concentration sensor if a malfunction of the gas concentration sensor or an interference source of the gas concentration sensor is detected. In some embodiments, the cleaning method is a method comprising an operation of the heater, e.g. with the maximum heating power and/or over a predetermined period of time. In doing so, water droplets on the temperature sensor field can evaporate or dirt particles can be burned, which could cause the malfunction of the gas concentration sensor.

In some embodiments, the cleaning method is initialized by a control unit. In some embodiments, the control unit initializes the cleaning method in response to the diagnosis signal.

In some embodiments, the temperature sensors are designed as thermoelectric temperature sensors. The temperature sensors can thus output an electronic signal, for example an analog or a digital signal, on the basis of the temperature at the temperature sensor. These are temperature sensors that comprise resistance thermometers or thermal elements, for example.

In some embodiments, the temperature sensor field is arranged in a temperature sensor field plane, wherein the heater is intersected by a subdivision plane running perpendicular to the temperature sensor field plane, wherein the regions of the temperature sensor field plane are arranged on one side and on the other side of the subdivision plane.

In some embodiments, the heater and/or the regions and/or the temperature sensor are arranged such that, during the generation of a heat flow by means of the heater, a uniform temperature distribution takes place and/or can be determined on one side and on the other side of the subdivision plane. In some embodiments, the heater is designed symmetrically to the subdivision plane. If further subdivision planes are provided, this subdivision plane is to be referred to as the first subdivision plane.

In some embodiments, the heater is intersected by a further subdivision plane running perpendicular to the temperature sensor field plane and perpendicular to the first subdivision plane, wherein the regions of the temperature sensor field plane are arranged on one side and on the other side of the further subdivision plane.

In some embodiments, the heater and/or the regions and/or the temperature sensor are arranged such that, during the generation of a heat flow by means of the heater, a uniform temperature distribution occurs and/or can be determined on one side and on the other side of the further subdivision plane. In some embodiments, the heater is designed symmetrically to the further subdivision plane.

In some embodiments, the gas or the gas mixture flows in the extension direction of the subdivision plane or in the extension direction of the first subdivision plane along the heater and/or the temperature sensor field.

In some embodiments, the first subdivision plane forms a plane of symmetry of the flow profile of a gas flow of the gas or the gas mixture flow of the gas mixture. This ensures a uniform temperature distribution on both sides of this subdivision plane.

In some embodiments, the first region is arranged on one side of the or the first subdivision plane, wherein the second region is arranged on the other side of the or the first subdivision plane.

In some embodiments, the first and second regions are arranged on the same side of the second subdivision plane. In some embodiments, the first and the second regions are arranged on a side of the subdivision plane facing away from the flow direction of the gas or the gas mixture. This ensures that a diagnosis of the gas concentration sensor is possible even in the event of a strong flow.

In some embodiments, the first and second regions are two regions separated from each other by at least one subdivision plane. In other words, the first region and the second region are separated from each other by the subdivision plane, by the first subdivision plane, by the second subdivision plane, or by both the first and the second subdivision planes.

In these embodiments, the gas concentration sensor is arranged in such a way that the gas concentration of the gas or the gas mixture is determined as statically as possible, i.e., without the relative movement of the gas or the gas mixture relative to the heater and/or the temperature sensor field plane, which is significant for the measurement. This is possible, for example, if the gas concentration sensor is arranged outside of a gas channel through which the gas or gas mixture flows, and/or is fluidically connected to the gas channel by means of a settling region.

Some embodiments include a gas concentration sensor having a heater, a temperature sensor field which has a plurality of regions, wherein each region comprises at least one temperature sensor, and means adapted to carry out one or more of the methods described herein. In some embodiments, the gas concentration sensor comprises other features that have been mentioned in relation to the method described above.

In some embodiments, the gas concentration sensor is fluidically coupled to a gas channel.

In some embodiments, the gas concentration sensor or the gas concentration sensor with the gas channel is/are provided as part of a fuel cell system. It is also conceivable to provide a vehicle or a stationary power generation system with such a fuel cell system.

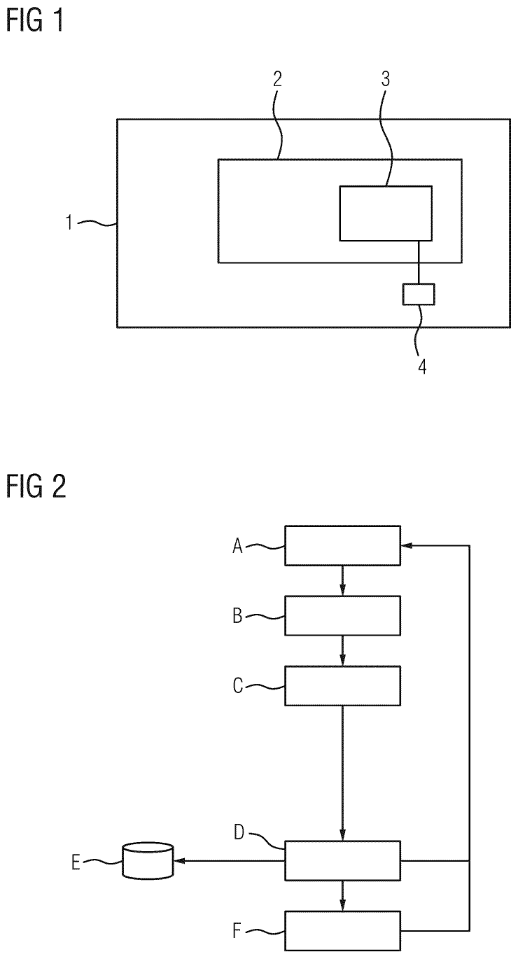

FIG. 1 shows an example motor vehicle 1 having a fuel cell system 2, a gas concentration sensor 3 incorporating teachings of the present disclosure as part of the fuel cell system 2, and a control unit 4 coupled to the gas concentration sensor 3. Alternatively, it is conceivable to use such a fuel cell system 2 with the associated gas concentration sensor 3 and the control unit 4 in a stationary power generation system.

FIG. 2 shows an example method incorporating teachings of the present disclosure. First, the start A of the method takes place. Then, by means of a heater, a heat flow B is generated.

In response to this, a temperature distribution C is determined on a temperature sensor field. This determination comprises determining temperature measurement values from different regions of the temperature sensor field. Subsequently, if a temperature measurement difference, which has been determined from the temperature measurement values, exceeds a predefined threshold value, a diagnosis D is carried out, which comprises detecting a malfunction or an interference source of a gas concentration sensor on which the method is carried out. If no malfunction or interference source is detected, the method is restarted, preferably time-shifted at regular intervals. Alternatively, it is conceivable to carry out the method when starting a fuel cell system to which the gas concentration sensor with which the method is carried out belongs. If a malfunction or an interference source is detected, then this information can be stored E on a storage medium, which can, for example, belong to a control unit. In addition or alternatively, an initialization of a cleaning method F can take place. The cleaning method can, for example, be a targeted heating of the gas concentration sensor by means of the heater. This allows dirt particles or water droplets to be removed from the temperature sensor field. After the cleaning method, the method is restarted and repeated until the interference source or the malfunction is eliminated.

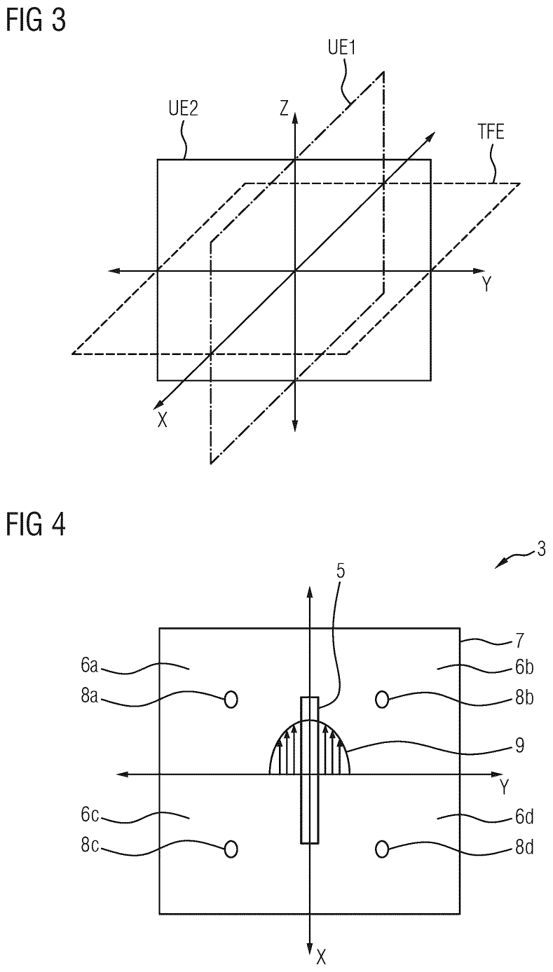

FIG. 3 shows three planes TFE, UE1, UE2 extending perpendicularly to each other. A temperature sensor field plane TFE extends in the X-Y plane, while a first subdivision plane UE1 extends in the X-Z plane and a further subdivision plane UE2 extends in the Y-Z plane. These planes TFE, UE1, UE2 are used to provide a better understanding of FIG. 4.

FIG. 4 shows an example gas concentration sensor incorporating teachings of the present disclosure. A view perpendicular to the temperature sensor field plane from FIG. 3 is shown. A temperature sensor field 7 can be seen, which extends in the temperature sensor field plane. The two subdivision planes from FIG. 3, the course of which are indicated in FIG. 4 by the X-axis and the Y-axis, intersect a heater 5 of the gas concentration sensor 3 and divide the temperature sensor field 7 of the gas concentration sensor 3 into four regions 6a to 6d, wherein each one of the regions has a temperature sensor 8a to 8d. The heater 5 is arranged symmetrically to the two subdivision planes from FIG. 3 to ensure a uniform temperature distribution. The heater 5 is an electric PTC heater. During the application of the method according to the invention by means of this gas concentration sensor 3, the gas or the gas mixture flows in a laminar manner in the extension direction of the X-axis. In other words, a flow profile 9, which is largely symmetrical to the first subdivision plane, is created. This results in a uniform temperature distribution on the temperature sensor field 7, which can be determined, for example, by means of the two temperature sensors 8a and 8b of the two regions 6a and 6b. In the case that these two temperature sensors 8a and 8b determine different temperature measurement values and a resulting temperature measurement value difference is above a predefined threshold value, a malfunction or an interference source of the gas concentration sensor is detected according to the invention.

The different features of the individual embodiments can also be combined with each other. The embodiments of FIGS. 1 to 4 in particular have no limiting character and serve to illustrate the concept of the disclosure.

### LIST OF REFERENCE SIGNS

1 Motor vehicle
2 Hydrogen system
3 Gas concentration sensor
4 Control unit
5 Heater
6a-6D Regions
7 Temperature sensor field
8a-8D Temperature sensor
9 Flow profile
UE1 First subdivision plane
UE2 Further subdivision plane
TFE Temperature sensor field plane
A Start
B Generating a heat flow
C Determining a temperature distribution
D Diagnosis
E Storing
F Initializing a cleaning method

What is claimed is:

1. A method for diagnosing a gas concentration sensor
configured to determine a concentration of a gas using a thermal conductivity and/or thermal capacity of the gas,
wherein the gas concentration sensor comprises a heater and a temperature sensor field with
a plurality of regions and at least one temperature sensor per region, the method comprising:
generating a heat flow with the heater;
determining a temperature distribution on the temperature sensor field using
the temperature sensors of the respective regions; and
diagnosing the gas concentration sensor using the determined temperature distribution.

2. The method as claimed in claim 1, further comprising:
determining a first temperature measurement value using a first temperature sensor in a first region;
determining a second temperature measurement value using a second temperature sensor in a second region;
determining a temperature measurement value difference based on the a first and second temperature measurement values; and
determining the temperature distribution using the temperature measurement value difference.

3. The method as claimed in claim 2, further comprising
detecting a malfunction or an interference source of the gas concentration sensor if the determined temperature measurement value difference exceeds a predefined threshold value.

4. The method as claimed in claim 3, wherein the threshold value depends on the position of the temperature sensors relative to each other and/or the position of the temperature sensors relative to the heater and/or the distance of the temperature sensors relative to the heater and/or the distance of the temperature sensors relative to each other.

5. The method as claimed in claim 1, further comprising generating a diagnosis signal using the determined temperature distribution.

6. The method as claimed in claim 3, further comprising initiating a cleaning method for cleaning the gas concentration sensor if the determined temperature measurement value difference exceeds a predefined threshold value.

7. The method as claimed in claim 1, wherein the temperature sensors comprises thermoelectric temperature sensors.

8. The method as claimed in claim 1,
wherein:
the temperature sensor field is arranged in a temperature Sensor field plane;
the heater is intersected by a subdivision plane running perpendicular to the temperature sensor field plane; and
the regions of the temperature sensor field plane are arranged on one side and on the other side of the subdivision plane.

9. The method as claimed in claim 8, wherein:
the heater is intersected by a further subdivision plane running perpendicular to the temperature sensor field plane and perpendicular to the first subdivision plane; and
the regions of the temperature sensor field plane are arranged on one side and on the other side of the further subdivision plane.

10. The method as claimed in claim 8, wherein the gas flows in the extension direction of the subdivision plane or in the extension direction of the first subdivision plane along the heater and/or the temperature sensor field.

11. The method as claimed in claim 10,
wherein:
the first region is arranged on one side of the or the first subdivision plane; and
the second region is arranged on the other side of the or the first subdivision plane.

12. The method as claimed in claim 11, wherein the first and second regions are arranged on the same side of the second subdivision plane.

13. The method as claimed in claim 8, wherein the first and second regions are two regions separated from each other by at least one subdivision plane.

14. A gas concentration sensor comprising:
a heater;
a temperature sensor field comprising a plurality of regions;
at least one temperature sensor in each region; and
a processor programmed to:
generate a heat flow with the heater;
determine a temperature distribution on the temperature sensor field using the temperature sensors of the respective regions; and
diagnose the gas concentration sensor using the determined temperature distribution.

15-16. (canceled)
